(19) United States
(12) Patent Application Publication
(10) Pub. No.: US 2021/0000877 A1
(43) Pub. Date: Jan. 7, 2021

(54) Method For Upregulation Of Thioredoxin Expression In Stem Cells

(71) Applicant: SAMSUNG LIFE PUBLIC WELFARE FOUNDATION, Seoul (KR)

(72) Inventors: Won Soon PARK, Seoul (KR); Yun Sil CHANG, Seoul (KR); Dong Kyung SUNG, Seoul (KR)

(21) Appl. No.: 16/977,524

(22) PCT Filed: Feb. 28, 2019
(86) PCT No.: PCT/KR2019/002408

§ 371 Date: Sep. 2, 2020

(30) Foreign Application Priority Data
Mar. 2, 2018 (KR) 10-2018-0025278
Feb. 27, 2019 (KR) 10-2019-0023053

(51) Int. Cl. A61K 35/28; C12N 5/0775; C12N 9/02; A61K 47/69; A61P 25/00

(52) U.S. Cl. CPC A61K 35/28; C12N 5/0662; C12Y 108/01008; A61K 47/6935; A61P 25/00; C12N 9/0051

(57) ABSTRACT

The present invention relates to a method for upregulation of thioredoxin expression in stem cells and, more particularly, to a method for upregulation of thioredoxin expression in stem cells, the method comprising a step of culturing stem cells in a hypoxic condition, the stem cells having an upregulated expression of thioredoxin by the same method, and a use of the stem cells in treatment of ischemic brain disease. When cultured in a hypoxic condition, stem cells are able to have increased expression of thioredoxin and stem cells that have an upregulated expression level of thioredoxin exhibiting an excellent therapeutic effect on ischemic brain disease can be obtained by the method. Thus, the stem cells can be usefully applied in the prevention or treatment of ischemic brain disease.

### FIELD

The present disclosure relates to a method of enhancing thioredoxin (TRX) expression in stem cells, and more particularly, to a method of enhancing thioredoxin expression in stem cells, the method including culturing stem cells in a hypoxic environment, stem cells in which thioredoxin expression is enhanced by the method, and a use of the stem cells for treating ischemic brain diseases.

### BACKGROUND

Thioredoxin (TRX) is a low-molecular-weight protein having a molecular weight of 10,000 to 13,000, and was discovered as a coenzyme that donates hydrogen ions to ribonucleotide reductase, which is an enzyme essential for DNA synthesis in E. coli. Thioredoxin has an active site called -Cys-Gly-Pro-Cys-, which has an oxidized form that forms a disulfide (S—S) bond between two cysteine residues and a reduced form that forms a dithiol (—SH—SH), and therefore, it is an intracellular redox-controlling factor.

Thioredoxin has been reported to have many biological activities, and for example, it has been reported that thioredoxin acts as a growth factor and removes hydrogen peroxide, which causes toxicity in cells, and affects NF-κB signaling activity in eukaryotic cells, thereby affecting cell death and tumors related to the signaling. Because of these activities, thioredoxin has recently attracted attention in the field of development of anticancer agents and antioxidant proteins that protect cells from damage.

Meanwhile, ischemic brain disease is a type of cerebrovascular disease and can be subdivided into thrombosis, embolism, transient ischemic attack, lacunes, and the like, and refers to a disease in which, mainly due to blood clots and embolism, pathological abnormalities occur in blood vessels that supply blood to the brain. When transient cerebral ischemia is induced in the cerebrum, the supply of oxygen and glucose is blocked, resulting in ATP reduction and edema in nerve cells, and ends up with extensive damage to the brain. Neuronal death occurs after a considerable amount of time after cerebral ischemia, which is referred to as delayed neuronal death.

Neuronal death caused by cerebral ischemia occurs by two main mechanisms. One is the excitatory neuronal death mechanism in which excessive glutamate is accumulated outside cells due to cerebral ischemia, and this glutamate enters the cells, eventually causing neuronal death due to excessive intracellular calcium accumulation. The other is the oxidative neuronal death mechanism caused by damage to DNA and the cytoplasm due to an increase in radicals in vivo resulting from sudden oxygen supply during ischemia-reperfusion.

Currently available drugs for the treatment of ischemic brain disease are antithrombotic agents, and it is reported that antiplatelet agents or anticoagulant agents such as ticlopidine, cilostazole, and prostacycline often cause headaches, palpitations, and side effects that burden the liver, and thus the use thereof is limited. In addition, FDA-approved commercially available tissue plasminogen activators induce rapid supply of oxygen and glucose by dissolving blood clots that cause cerebral ischemia, but do not directly protect nerve cells, and thus early use thereof is required, and due to the characteristics thereof as a thrombolytic agent, excessive use or frequent use leads to thinning of the blood vessel wall, thus eventually inducing hemorrhagic cerebrovascular disease (Int J Stroke. 2014 April; 9(3):349-55).

To date, in addition to the aforementioned drugs, research on the development of new therapeutic drugs based on the pathogenesis of ischemic brain disease has been conducted in various ways, but there have been few substances that inhibit neuronal death caused by cerebral ischemia.

### SUMMARY

### Technical Problem

The present invention has been made to address the above-described conventional problems, and the inventors of the present invention observed that, when stem cells were cultured under ischemic hypoxic conditions, the expression of thioredoxin was specifically significantly increased, unlike under normal conditions, and confirmed a neuronal death inhibitory effect induced thereby, and thus completed the present invention based on these findings.

Therefore, an object of the present invention is to provide a method of enhancing thioredoxin (TRX) expression in stem cells and stem cells in which thioredoxin expression is enhanced by the method.

Another object of the present invention is to provide a pharmaceutical composition for treating an ischemic brain disease, including the stem cells in which thioredoxin expression is enhanced.

Still another object of the present invention is to provide a complex for thioredoxin delivery, including thioredoxin (TRX) and polydimethylsiloxane (PDMS) nanoparticles.

Yet another object of the present invention is to provide a pharmaceutical composition for treating an ischemic brain disease, including the complex.

However, technical problems to be solved by the present invention are not limited to the above-described technical problems, and other unmentioned technical problems will become apparent from the following description to those of ordinary skill in the art.

### Technical Solution

According to an aspect of the present invention, there is provided a method of enhancing thioredoxin (TRX) expression in a stem cell, including culturing stem cells in a hypoxic environment.

The present invention also provides a stem cell in which thioredoxin expression is enhanced by the method.

In one embodiment of the present invention, the hypoxic environment may be an environment in which 0.5% to 10% oxygen is supplied.

In another embodiment of the present invention, the stem cell may be selected from the group consisting of a mesenchymal stem cell, a human tissue-derived mesenchymal stromal cell, a human tissue-derived mesenchymal stem cell, a multipotent stem cell, and an amniotic epithelial cell.

In another embodiment of the present invention, the mesenchymal stem cell may be derived from an umbilical cord, umbilical cord blood, bone marrow, fat, muscle, nerves, skin, amniotic membranes, placentas, amniotic fluid, or tonsils.

The present invention also provides a pharmaceutical composition for treating an ischemic brain disease, including the stem cell in which thioredoxin expression is enhanced.

In one embodiment of the present invention, the ischemic brain disease may be any one selected from the group consisting of neonatal hypoxic ischemic brain injury, stroke, cerebral infarction, cerebral ischemia, thrombosis, embolism, transient ischemic attack, lacunes, cerebral hemorrhage, head trauma, cerebral circulation metabolic disorders, brain function coma, traumatic brain injury, and hypoxic brain injury.

The present invention also provides a method of treating an ischemic brain disease, including administering, to an individual, a pharmaceutical composition for treating an ischemic brain disease, including the stem cell in which thioredoxin expression is enhanced.

The present invention also provides a use of the pharmaceutical composition for treating an ischemic brain disease.

The present invention also provides a complex for thioredoxin delivery, including thioredoxin (TRX) and polydimethylsiloxane (PDMS) nanoparticles.

In one embodiment of the present invention, the nanoparticles may be prepared by mixing tetraethyl orthosilicate (TEOS) and dimethyldiethoxysilane (DMDES), followed by a condensation reaction in the presence of a base catalyst.

The present invention also provides a pharmaceutical composition for treating an ischemic brain disease, including the complex.

The present invention also provides a method of treating an ischemic brain disease, including administering the pharmaceutical composition including the complex to an individual.

The present invention also provides a use of the pharmaceutical composition including the complex for treating an ischemic brain disease.

### Advantageous Effects

The inventors of the present invention confirmed that, when stem cells were cultured under a hypoxic condition, the expression of thioredoxin was specifically significantly enhanced only under a hypoxic condition, unlike under normal conditions, confirmed that cell death was inhibited by thioredoxin in injury-induced nerve cells in an ischemic hypoxic environment, and confirmed the therapeutic effect of thioredoxin through a carrier upon intraventricular administration in a neonatal hypoxic ischemic encephalopathy in vivo model. Thus, by culturing stem cells in a hypoxic environment, thioredoxin expression can be enhanced, and through the method, stem cells, which have excellent therapeutic efficacy for ischemic brain diseases and in which thioredoxin expression is enhanced, can be obtained, and thus the stem cells can be effectively used as a use for the prevention or treatment of ischemic brain diseases.

### BRIEF DESCRIPTION OF DRAWINGS

FIG. 1A illustrates the results confirming 61 proteins (OGD abundance) from a culture medium of stem cells (OGD) cultured under a hypoxic condition, the 61 proteins exhibiting enhanced expression as compared to control stem cells (NC), and subcellular locations thereof.

FIG. 1B illustrates the results showing that thioredoxin expression was significantly increased in stem cells (OGD) cultured under a hypoxic condition, as compared to a control (NC).

FIG. 2A illustrates the results showing that, as a result of exposing nerve cells derived from the cerebral cortex of white mice fetuses to an oxygen concentration of 1% for 1 hour to induce cell injury, and then treating the cells with thioredoxin (OGD thioredoxin), cell death was significantly inhibited as compared to a group not treated with thioredoxin (OGD control).

FIG. 2B illustrates the results of measuring the cell survival and reactive oxygen species (ROS) levels of nerve cells cultured under the same conditions as those in FIG. 2A after being treated with various concentrations (1, 10, 100, 500, or 1,000 ng) of thioredoxin or treated with mesenchymal stem cells (MSCs).

FIG. 3A illustrates a process of synthesizing a carrier for the delivery of thioredoxin into neurons.

FIG. 3B illustrates in vitro results showing intracellular uptake of the carrier.

FIG. 3C illustrates ex vivo results showing the uptake of the carrier into nerve cells and results showing the uptake of the carrier into brain cells upon administration to the ventricles of rats.

FIG. 4 illustrates the results showing a therapeutic effect of thioredoxin on improving damaged brain tissue when was intraventricularly administered through the carrier in a neonatal hypoxic ischemic encephalopathy in vivo model.

### DETAILED DESCRIPTION

The inventors of the present invention observed that, when stem cells were cultured under ischemic hypoxic conditions, the expression of thioredoxin was specifically significantly increased, unlike under normal conditions, and confirmed a neuronal death inhibitory effect induced thereby, and thus completed the present invention based on these findings.

Therefore, the present invention provides a method of enhancing thioredoxin (TRX) expression in a stem cell, including culturing stem cells in a hypoxic environment.

The present invention also provides a stem cell in which thioredoxin expression is enhanced by the method.

The inventors of the present invention confirmed through examples that, when stem cells were cultured in a hypoxic environment, thioredoxin expression was significantly enhanced. More specifically, when a culture medium of stem cells cultured under a hypoxic condition (culture at an oxygen concentration of 5% for 1 hour, followed by culture under a normal condition for 6 hours) or under a normal condition (culture under a normal condition for 7 hours) was collected and cultured under a hypoxic condition, 61 proteins were confirmed as a result of examining proteins exhibiting specifically enhanced expression, and it was confirmed that, among these, the expression of thioredoxin protein was significantly increased (see Example 1).

In the present invention, the hypoxic environment may be an environment in which 0.5% to 10% oxygen is supplied, more preferably an environment in which 1% to 5% oxygen is supplied.

The term “stem cell” as used herein refer to an undifferentiated cell which has a self-replication ability and the ability to differentiate into two or more different types of cells. The stem cell of the present invention may be an autologous or allogenic stem cell, and may be derived from any type of animal including humans and non-human mammals, and the stem cell may be derived from an adult or an embryo, but the present invention is not limited thereto.

In the present invention, the stem cell may be selected from the group consisting of a mesenchymal stem cell, a human tissue-derived mesenchymal stromal cell, a human tissue-derived mesenchymal stem cell, a multipotent stem cell, and an amniotic epithelial cell, and more preferably, the stem cell of the present invention may be a mesenchymal stem cell. The mesenchymal stem cell may be derived from an umbilical cord, umbilical cord blood, bone marrow, fat, muscle, nerves, skin, amniotic membranes, placentas, amniotic fluid, or tonsils, but the origin of the mesenchymal stem cell is not limited as long as it is derived from human body tissue.

Another embodiment of the present invention provides a pharmaceutical composition for treating an ischemic brain disease, including the stem cell with enhanced thioredoxin expression.

In one embodiment of the present invention, the effect of thioredoxin, exhibiting enhanced expression in stem cells in a hypoxic environment, on nerve cells was verified. More specifically, nerve cells extracted from the cerebral cortex of white mice fetuses were exposed to an oxygen concentration of 1% for 1 hour to induce injury, followed by thioredoxin treatment, and cell survival was analyzed. As a result, it was confirmed that, compared to nerve cells not treated with thioredoxin, neuronal death was significantly inhibited, and it was also confirmed that this effect was exhibited in proportion to the treatment concentration of thioredoxin and the concentration of reactive oxygen species was reduced (see Example 2).

In another embodiment of the present invention, a carrier capable of intracellularly delivering thioredoxin was synthesized, and it was confirmed that the carrier was absorbed into nerve cells, and furthermore, when a carrier including thioredoxin was intraventricularly administered in a neonatal hypoxic ischemic encephalopathy in vivo model, a therapeutic effect of significantly improving damaged brain tissue was confirmed.

These results show a therapeutic effect of thioredoxin on ischemic brain diseases, and also demonstrates that stem cells with enhanced thioredoxin expression by being cultured in an ischemic hypoxic environment can be effectively used in the treatment of ischemic brain diseases.

The ischemic brain disease may be any one selected from the group consisting of neonatal hypoxic ischemic brain injury, stroke, cerebral infarction, cerebral ischemia, thrombosis, embolism, transient ischemic attack, lacunes, cerebral hemorrhage, head trauma, cerebral circulation metabolic disorders, brain function coma, traumatic brain injury, and hypoxic brain injury, but the present invention is not limited thereto.

Another embodiment of the present invention provides a complex for thioredoxin delivery, including thioredoxin (TRX) and polydimethylsiloxane (PDMS) nanoparticles.

The nanoparticles may be prepared by mixing tetraethyl orthosilicate (TEOS) and dimethyldiethoxysilane (DMDES), followed by a condensation reaction in the presence of a base catalyst, but the preparation method is not limited thereto.

Another embodiment of the present invention provides a pharmaceutical composition for treating an ischemic brain disease, including the complex.

The pharmaceutical composition of the present invention may further include one or more known adjuvant ingredients having the effect of treating an ischemic brain disease along with a stem cell with enhanced thioredoxin expression or the complex.

The pharmaceutical composition of the present invention may be used alone for the treatment of an ischemic brain disease, or may be used in combination with surgery, radiotherapy, hormone treatment, chemotherapy, and methods using a biological response modifier.

The composition of the present invention may further include a suitable carrier commonly used in preparation of a pharmaceutical composition. The pharmaceutically acceptable carrier, which is commonly used in preparations, may include, but is not limited to, a saline solution, sterilized water, Ringer's solution, buffered saline, cyclodextrin, a dextrose solution, a maltodextrin solution, glycerol, ethanol, and liposomes, and as needed, may further include other general additives such as an antioxidant and buffer. In addition, the composition may be formulated as injectable preparations such as an aqueous solution, a suspension, and an emulsion, pills, capsules, granules, or tablets by additionally adding a diluent, a dispersant, a surfactant, a binder, a lubricant, or the like. Suitable pharmaceutically acceptable carriers may be preferably formulated according to respective ingredients using methods disclosed in Remington's document regarding suitable pharmaceutically acceptable carriers and formulations. The pharmaceutical composition of the present invention is not particularly limited in terms of formulation, but may be formulated as an injection, an inhalant, a preparation for external application to the skin, or the like.

The pharmaceutical composition of the present invention may be administered orally or parenterally (for example, intravenously, subcutaneously, intraperitoneally or locally) according to the desired method, and a dose thereof may vary depending on the condition and body weight of a patient, the severity of disease, drug form, administration route, and administration time, but may be appropriately selected by those of ordinary skill in the art.

The pharmaceutical composition of the present invention is administered in a pharmaceutically effective amount. In the present invention, “pharmaceutically effective amount” refers to an amount sufficient to treat or diagnose diseases at a reasonable benefit/risk ratio applicable to medical treatment or diagnosis, and an effective dosage level may be determined according to factors including type of diseases of patients, the severity of disease, the activity of drugs, sensitivity to drugs, administration time, administration routes, excretion rate, treatment period, and simultaneously used drugs, and other factors well known in the medical field. The pharmaceutical composition according to the present invention may be administered as an individual therapeutic agent or in combination with other therapeutic agents, and may be administered sequentially or simultaneously in combination therewith, and may be administered in a single dose or multiple doses. It is important to administer the pharmaceutical composition in the minimum amount that enables achievement of the maximum effects without side effects in consideration of all the above-described factors, and this may be easily determined by those of ordinary skill in the art.

The present invention also provides a method of treating an ischemic brain disease, including administering, to an individual, a pharmaceutical composition including the stem cell with enhanced thioredoxin expression.

In the present invention, “individual” refers to a subject in need of treatment of a disease, and more particularly, means mammals such as humans or non-human primates, mice, rats, dogs, cats, horses, and cows.

The present invention also provides a use of the pharmaceutical composition for treating an ischemic brain disease.

Hereinafter, exemplary examples will be described to aid in understanding of the present invention. However, these examples are provided only to facilitate the understanding of the present invention and are not intended to limit the scope of the present invention.

### EXAMPLES

### Example 1. Investigation of Proteins Secreted by Stem Cells Under Hypoxic Condition and Discovery of Thioredoxin

The inventors of the present invention conducted this experiment to develop a stem cell therapeutic agent for ischemic brain diseases, which is an ischemic hypoxic environment.

To this end, first, proteins secreted from stem cells exposed to hypoxic conditions were examined. More specifically, stem cells were cultured for 1 hour at an oxygen concentration of 5%, and then cultured for 6 hours under a normal condition, and then the culture medium of the cells was collected, and as a control, the same type of stem cells were cultured for 7 hours only under a normal condition, and then the culture medium of the cells was collected. Next, each collected culture medium was centrifuged at 3,000 rpm for 30 minutes to remove debris in the culture medium, and then a process of concentrating the culture medium was performed for protein analysis. Subsequently, fractionation was performed after trypsin digestion, and mass analysis was performed to analyze proteins exhibiting increased expression in the stem cells exposed to a hypoxic condition, as compared to control stem cells.

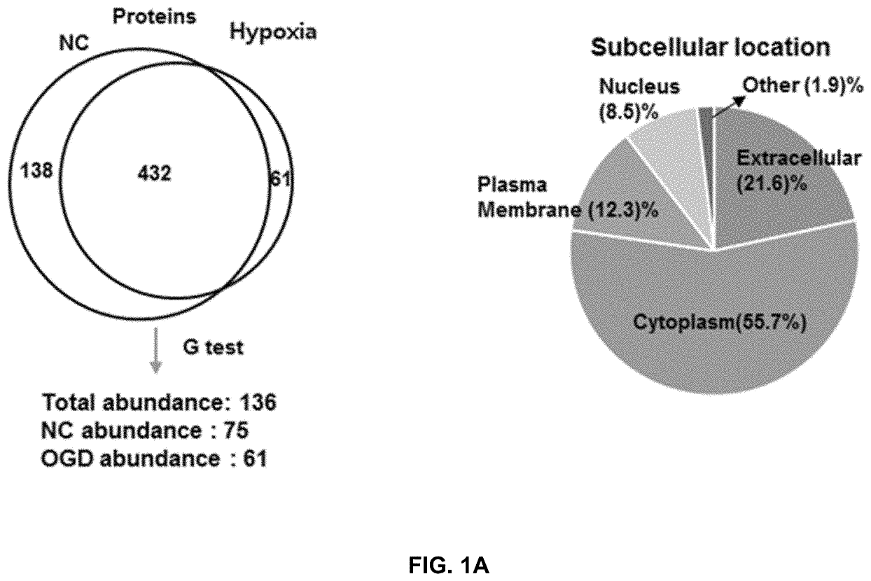

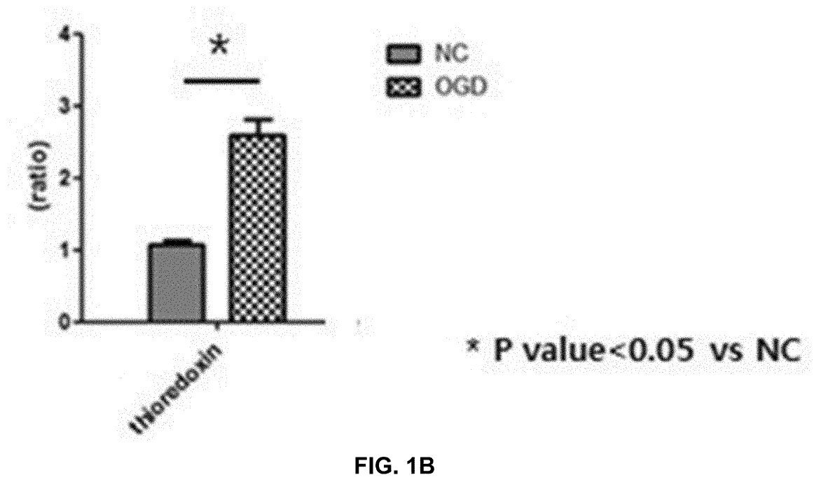

As a result, as illustrated in FIG. 1A, increases in the expression of a total of 61 proteins in a hypoxic condition were confirmed, and as a result of examining subcellular locations of these proteins, Cytoplasm 55.7%, Extracellular 21.6%, Plasma membrane 12.3%, Nucleus 8.5%, and Other 1.9% were shown. The inventors of the present invention finally selected thioredoxin from among the 61 proteins, as a protein satisfying all of the following conditions: 1) an antioxidant protein being researched as a therapeutic target for an ischemic hypoxic brain disease; ii) a synthetic protein is much cheaper than a recombinant protein; and iii) easy to develop various types of carriers, and then further analysis was performed. The results of a change in the expression of thioredoxin in a hypoxic environment are shown in FIG. 1B.

### Example 2. Verification of Neuronal Death Inhibitory Effect of Thioredoxin in Ischemic Hypoxic Environment

Based on the results of Example 1, the inventors of the present invention attempted to verify the effect of the thioredoxin on nerve cells in an ischemic hypoxic environment. To this end, the neuronal death inhibitory effect of thioredoxin in an ex vivo model with neuronal death induced by hypoxia was examined.

More specifically, fetuses were extracted from white mice at a gestational age of 18.5 days, the cerebral cortex was separated, and nerve cells were cultured, and at this time, a nerve cell culture medium was used and the nerve cells were allowed to undergo adhesion and stabilization for 7 days. Subsequently, the nerve cells were exposed to a 1% oxygen condition for 60 minutes to induce cell injury, and then the nerve cells with induced cell death were treated with thioredoxin to observe a cell death inhibitory effect, and cell survival was analyzed using a cell count kit (Dojindo, Korea).

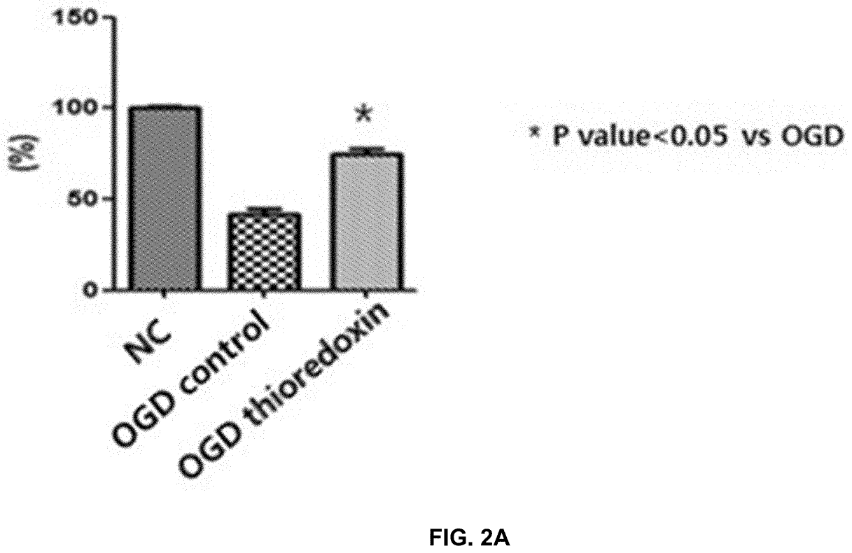

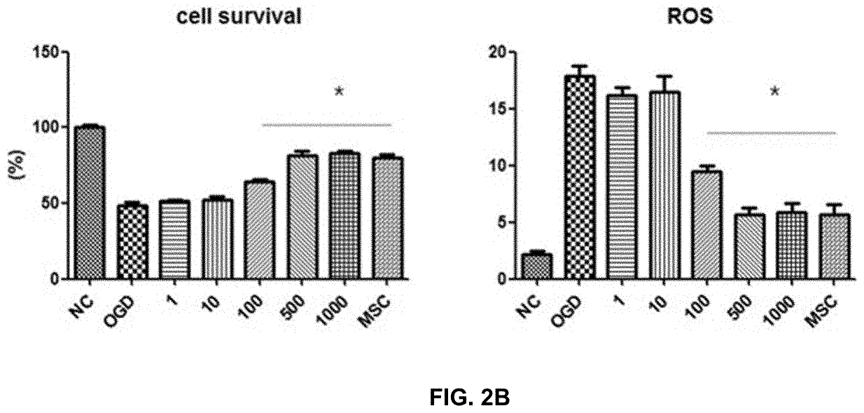

As a result, as illustrated in FIG. 2A, it was confirmed that, in the case of nerve cells exposed only to a hypoxic condition and not treated with thioredoxin (OGD control), neuronal death was significantly induced as compared to a control (NC) cultured under a normal condition, whereas cell death was inhibited at a significant level in nerve cells treated with thioredoxin (OGD thioredoxin).

In addition, nerve cells cultured under the same conditions as described above were treated with various concentrations (1, 10, 100, 500, or 1,000 ng) of thioredoxin or treated with mesenchymal stem cells (MSCs), and then cell survival was measured in the same manner as described above. In addition, a reactive oxygen species assay kit was used to measure the amount of active oxygen change, which was used as a tool for evaluating therapeutic efficacy.

As a result, as illustrated in FIG. 2B, as the treatment concentration of thioredoxin increased, cell death was inhibited and a reactive oxygen species (ROS) reduction pattern was exhibited, and in particular, it was confirmed that, when the treatment concentration is 500 ng or more, the same cell survival and ROS reduction effects as those in the case of treatment with mesenchymal stem cells themselves were exhibited.

These results demonstrate the neuronal death inhibitory effect of thioredoxin secreted by stem cells in a hypoxic ischemic environment.

### Example 3. Verification of Delivery of Thioredoxin to Neurons Through Carrier and In Vivo Therapeutic Effect Thereof on Ischemic Brain Disease

3-1. Thioredoxin Carrier Synthesis

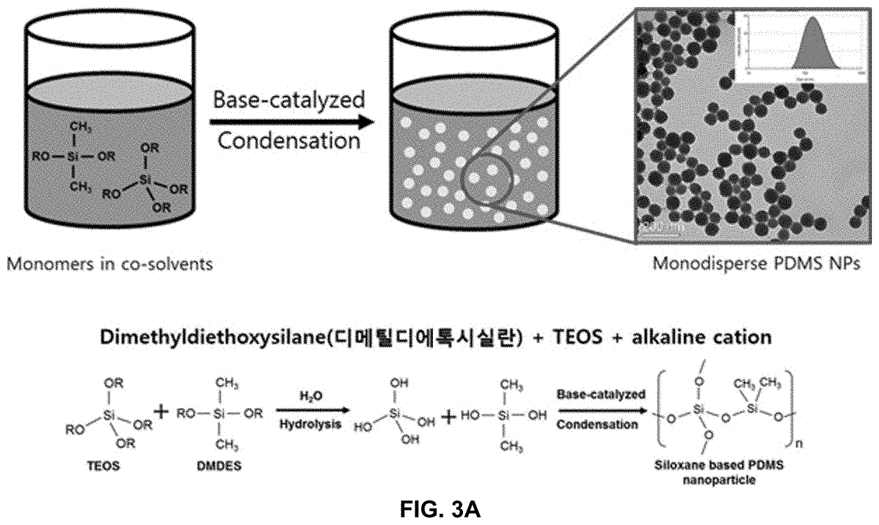

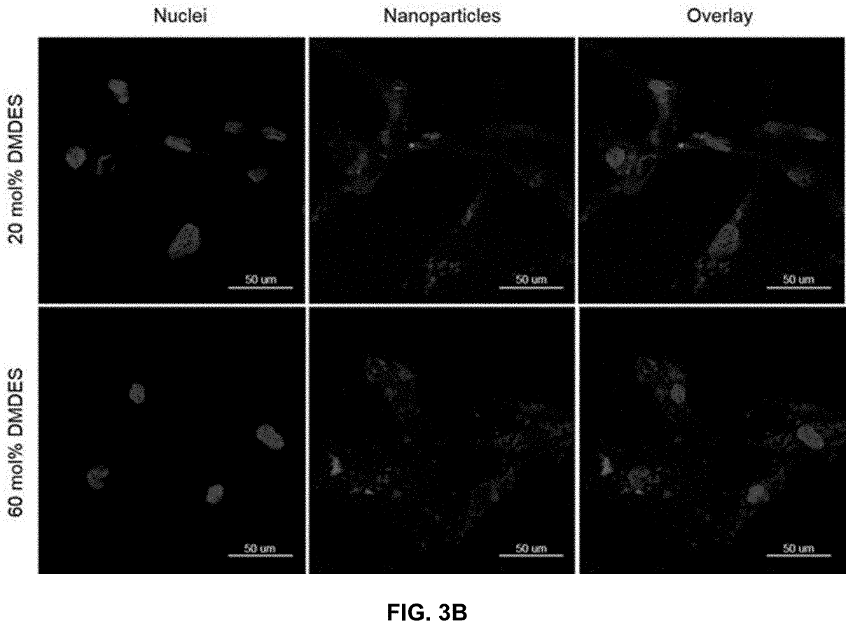

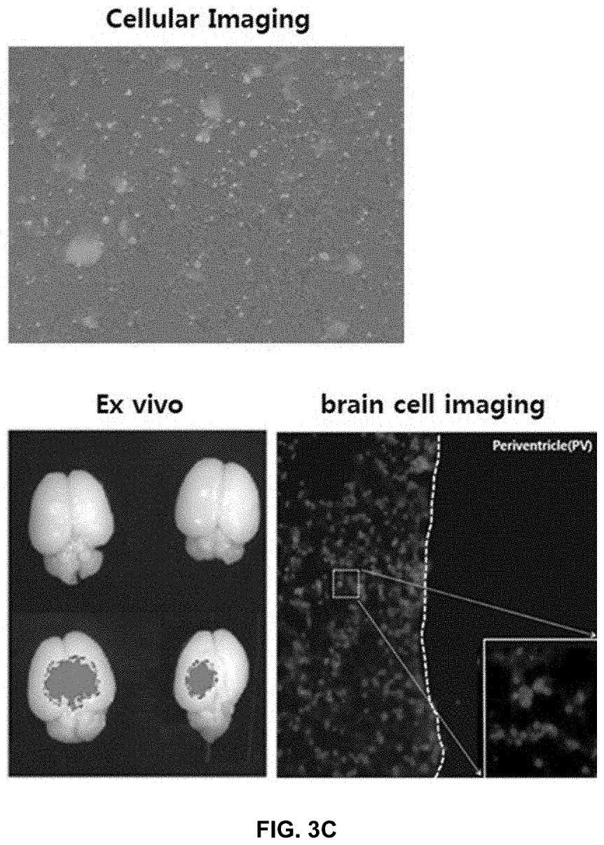

The inventors of the present invention synthesized a carrier capable of intracellularly delivering thioredoxin according to the following method. More specifically, as illustrated in FIG. 3A, tetraethyl orthosilicate (TEOS) and dimethyldiethoxysilane (DMDES) were mixed and subjected to hydrolysis, and then a siloxane-based PDMS nanoparticle carrier was prepared through a condensation reaction in the presence of a base catalyst. Subsequently, it was examined whether the carrier prepared by the method was intracellularly absorbed.

3-2. Confirmation of Intracellular Uptake of Carrier

To verify the intracellular uptake of the carrier prepared according to the method of Example 3-1, the following experiment was conducted. More specifically, a 37° C. thermostatic bath was heated and a frozen cell tube was rapidly melted in the thermostatic bath for 2-3 minutes, and then when the cells were completely melted, the cells were centrifuged at a speed of 300×g and room temperature for 5 minutes. After the centrifugation, the supernatant was removed, and a cell pellet was added to a DMEM culture medium and centrifuged once again under the same conditions, and then the supernatant was removed and the cell pellet was added to a culture medium and re-suspended. Next, the number of cells was measured using a LUNA-FL™ double-fluorescent cell counter, and then the cells were seeded at a density of 4,000 cells/cm² and incubated in an incubator. When the cells reached about 50% confluency, 1 μl of a tetramethylrhodamine (TRITC)-containing carrier was added to the culture medium of the cultured cells and cultured for 24 hours, and then it was observed through a fluorescence microscope whether the carrier was delivered into the cells.

As a result, as illustrated in FIG. 3B, when the carrier was treated at two concentrations, red fluorescence was observed in the cytoplasm of all the cases, from which it was confirmed that the carrier was efficiently absorbed into the cells.

3-3. Confirmation of Delivery of Carrier into Nerve Cells and Brain

Based on the results of Example 3-2, the inventors of the present invention further attempted to verify whether the carrier was delivered into nerve cells and the brain.

First, to confirm the presence or absence of delivery into nerve cells, fetuses were extracted from white mice at a gestational age of 18 days, and the cerebral cortex was separated, followed by trypsin treatment and pipetting, to be separated into single cells. Subsequently, the cells were counted and seeded at a density of 20,000 cells/cm² and incubated in an incubator. When the cells reached about 50% confluency, 1 μl of a tetramethylrhodamine-containing carrier was added to the culture medium of the cultured cells and cultured for 24 hours, and then it was observed through a fluorescence microscope whether the carrier was delivered into the cells.

As a result, as can be seen from the upper image of FIG. 3C, it was confirmed that the carrier was absorbed into nerve cells.

Furthermore, to verify whether the carrier is absorbed into nerve cells even when administered intraventricularly, 10 μl of the tetramethylrhodamine-containing carrier was administered intraventricularly to 7-day-old rats. After 24 hours, the brain was extracted from each rat and fluorescence was observed through optical image analysis. In addition, on the other hand, an OCT compound was sprayed around the brain tissue extracted by the method and then frozen, the brain tissue was cut to 10 μm using a cryotome, and then brain tissue sections were attached to slide glasses, followed by observation of fluorescence in the brain tissue through a fluorescence microscope.

As a result, as can be seen from the lower image of FIG. 3C, when the carrier was administered intraventricularly, fluorescence was observed in the administration site, and as a result of observing the brain tissue through a fluorescence microscope, red fluorescence was observed, through which it can be seen that the carrier is absorbed into brain cells.

3-4. Confirmation of Effect of Treating Ischemic Brain Disease by Administration of Thioredoxin Carrier

Based on the results of Examples 3-2 and 3-3, the inventors of the present invention attempted to verify whether, when thioredoxin is administered intraventricularly along with the carrier, a substantial effect of treating an ischemic brain disease is exhibited.

To this end, the right carotid arteries of 7-day-old male SD rats were tied and then exposed to a hypoxic environment, i.e., an oxygen concentration of 8%, for 2 hours and 10 minutes to produce a hypoxic ischemic encephalopathy (HIE) in vivo model. Subsequently, 10 μl of a thioredoxin-containing carrier was administered into the right ventricle, and after 72 hours, the brain was extracted and triphenyl tetrazolium chloride (TTC) staining was performed to confirm the extent to which the brain tissue was damaged. In the TTC staining, a reagent for staining mitochondrial dehydrogenase was used, and when nerve cells die, they appear white since mitochondria cannot secrete dehydrogenase, and normal cells appear red. The staining was performed by dissolving TTC powder in 37° C. physiological saline to prepare a 2% w/v TTC solution, and then immersing the brain tissue in the solution and culturing the tissue at 37° C. for 15 minutes to 30 minutes.

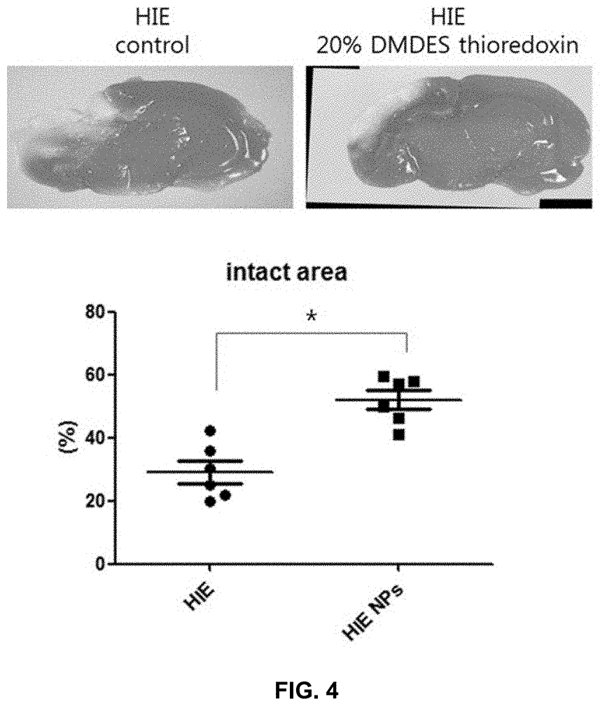

As a result, as illustrated in FIG. 4, it was confirmed that, as compared to a control that was not administered the thioredoxin-containing carrier, the case administered the thioredoxin-containing carrier (20% DMDES thioredoxin) exhibited a significant therapeutic effect of improving damaged brain tissue.

The foregoing description of the present invention is provided for illustrative purposes, and it will be understood by those of ordinary skill in the art to which the present invention pertains that the invention may be easily modified into many different forms without departing from the technical spirit or essential characteristics of the present invention. It is therefore to be understood that the above-described embodiments are illustrative in all aspects and not restrictive.

### INDUSTRIAL APPLICABILITY

According to the method according to the present invention, thioredoxin expression in stem cells can be significantly enhanced, and not only the effect of the thioredoxin on inhibiting neuronal death, but also the therapeutic effect thereof when thioredoxin is administered intraventricularly through a carrier in a neonatal hypoxic ischemic encephalopathy in vivo model have been experimentally confirmed, and thus stem cells cultured in a hypoxic environment, in which thioredoxin expression is enhanced by the method according to the present invention, can be effectively used in the field of treatment of ischemic brain diseases.

1. A method of enhancing thioredoxin (TRX) expression in a stem cell, the method comprising culturing stem cells in a hypoxic environment.

2. The method of claim 1, wherein the hypoxic environment is an environment in which 0.5% to 10% oxygen is supplied.

3. The method of claim 1, wherein the stem cell is a stem cell selected from the group consisting of a mesenchymal stem cell, a human tissue-derived mesenchymal stromal cell, a human tissue-derived mesenchymal stem cell, a multipotent stem cell, and an amniotic epithelial cell.

4. The method of claim 3, wherein the mesenchymal stem cell is derived from an umbilical cord, umbilical cord blood, bone marrow, fat, muscle, nerves, skin, amniotic membranes, placentas, amniotic fluid, or tonsils.

5. A stem cell in which thioredoxin (TRX) expression is enhanced by the method of claim 1.

6. The stem cell of claim 5, wherein the stem cell is a stem cell selected from the group consisting of a mesenchymal stem cell, a human tissue-derived mesenchymal stromal cell, a human tissue-derived mesenchymal stem cell, a multipotent stem cell, and an amniotic epithelial cell.

7. The stem cell of claim 6, wherein the mesenchymal stem cell is derived from an umbilical cord, umbilical cord blood, bone marrow, fat, muscle, nerves, skin, amniotic membranes, placentas, amniotic fluid, or tonsils.

8. A method of treating an ischemic brain disease, comprising:
administering to a subject in need thereof an effective amount of the stem cell of claim 5.

9. The method of claim 8, wherein the stem cell is a stem cell selected from the group consisting of a mesenchymal stem cell, a human tissue-derived mesenchymal stromal cell, a human tissue-derived mesenchymal stem cell, a multipotent stem cell, and an amniotic epithelial cell.

10. The method of claim 9, wherein the mesenchymal stem cell is derived from an umbilical cord, umbilical cord blood, bone marrow, fat, muscle, nerves, skin, amniotic membranes, placentas, amniotic fluid, or tonsils.

11. The method of claim 8, wherein the ischemic brain disease is any one selected from the group consisting of neonatal hypoxic ischemic brain injury, stroke, cerebral infarction, cerebral ischemia, thrombosis, embolism, transient ischemic attack, lacunes, cerebral hemorrhage, head trauma, cerebral circulation metabolic disorders, brain function coma, traumatic brain injury, and hypoxic brain injury.

12. A complex for thioredoxin delivery, the complex comprising thioredoxin (TRX) and polydimethylsiloxane (PDMS) nanoparticles.

13. The complex of claim 12, wherein the nanoparticles are prepared by mixing tetraethyl orthosilicate (TEOS) and dimethyldiethoxysilane (DMDES), followed by a condensation reaction in the presence of a base catalyst.

14. A method of treating an ischemic brain disease, comprising:
administering to a subject in need thereof an effective amount of the complex of claim 12.

15-18. (canceled)
